SEQUENCE LISTING (6 sequences)

SEQ ID NO: 1 - DNA, 26 bp, Artificial Sequence
  primer
attctagtggatcttttcaatgacca

SEQ ID NO: 2 - DNA, 24 bp, Artificial Sequence
  primer
cgtcagaacaaggacatttttcat

SEQ ID NO: 3 - DNA, 28 bp, Artificial Sequence
  probe
tacttggaggacaatgtcacagcccctg

SEQ ID NO: 4 - DNA, 20 bp, Artificial Sequence
  primer
ccacactgtgcccatctacg

SEQ ID NO: 5 - DNA, 26 bp, Artificial Sequence
  primer
aggatcttcatgaggtagtcagtcag

SEQ ID NO: 6 - DNA, 26 bp, Artificial Sequence
  probe
ntgcccncccccatgccatcctgcgt

(19) United States
(12) Patent Application Publication
(10) Pub. No.: US 2011/0195415 A1
(43) Pub. Date: Aug. 11, 2011

(54) GUANYLYL CYCLASE C QRT-PCR

(75) Inventors: Scott A. Waldman, Ardmore, PA (US); Theresa Hyslop, Glenside, PA (US); Inna Chervoneva, Philadelphia, PA (US); Stephanie Schulz, West Chester, PA (US)

(73) Assignee: THOMAS JEFFERSON UNIVERSITY, Philadelphia, PA (US)

(21) Appl. No.: 12/997,545

(22) PCT Filed: May 13, 2009
(86) PCT No.: PCT/US09/43857

§ 371 Date: Apr. 22, 2011

Related U.S. Application Data
(60) Provisional application No. 61/052,915, filed on May 13, 2008.

(51) Int. Cl. C12Q 1/68; G06F 19/00

(52) U.S. Cl. 435/6.12; 702/19

(57) ABSTRACT

Methods, kits, compositions and systems for detecting the level of GCC encoding mRNA present in a sample using quantitative (q) RT-PCR are disclosed. The methods, kits, compositions and systems may be used to detect metastasis in patients diagnosed with primary colorectal, gastric or esophageal cancer, to predict the risk of occurrence of relapse in patients diagnosed with primary colorectal, gastric or esophageal cancer, and to diagnose Barrett's esophagus.

### FIELD OF THE INVENTION

The present invention related to methods using and kits, compositions and systems used in quantitative RT-PCR of guanylyl cycles C mRNA to identify metastatic colorectal, gastric or esophageal cancer, to predict recurrence risk in colorectal, gastric or esophageal cancer patients, and to diagnose Barrett's esophagus.

### BACKGROUND OF THE INVENTION

This application claims priority to U.S. provisional application Ser. No. 61/052,915, filed May 13, 2008, which is incorporated herein by reference.

Metastasis of tumor cells to regional lymph nodes is the single most important prognostic factor in patients with colorectal cancer.^{1, 2} Recurrence rates increase from approximately 25% in patients with lymph nodes free of tumor cells by histopathology (pN0) to approximately 50% in patients with ≧4 lymph nodes harboring metastases.^{3, 4} Adjuvant chemotherapy improves disease-free and overall survival in patients with histopathologically evident lymph node metastases, but its role in pN0 patients remains unclear.⁵⁻⁹

Given the established relationship between lymph node metastasis and prognosis, recurrence in a substantial minority of pN0 patients suggests the presence of occult lymph node metastases [pN0(mol+)³] in regional lymph nodes that escape histopathological detection.^{1, 2} Conversely, pN0 patients who are free of lymph node metastases may be at lowest risk for developing recurrent disease. Thus, a more accurate assessment of occult metastases in regional lymph nodes in pN0 patients could improve risk stratification in this clinically heterogeneous population. In addition to enabling more accurate prognostication, precise evaluation of lymph node metastases could identify pN0 patients who might benefit from adjuvant chemotherapy.

Guanylyl cyclase C (GCC, also referred to as GUCY2C), an intestinal tumor suppressor, is the receptor for the paracrine hormones guanylin and uroguanylin, gene products frequently lost early in colon carcinogenesis.^{11, 12} The nucleic acid and amino acid sequences are known (see de Sauvage et al. 1991 J. Biol. Chem. 266 (27): 17912, which is incorporated herein by reference. Loss of hormone expression, with dysregulated GCC signaling contributes to neoplastic transformation through unrestricted proliferation, crypt hypertrophy, metabolic remodeling and genomic instability.¹² Selective expression by intestinal epithelial cells normally and universal over-expression by intestinal tumor cells¹³⁻¹⁵, reflecting receptor supersensitization in the context of ligand deprivation, suggest that GCC is a specific molecular marker for metastatic colorectal cancer.¹⁶⁻¹⁸ In a previous retrospective study, we found that GCC messenger RNA (mRNA) expression quantified by the reverse transcriptase-polymerase chain reaction (RT-PCR) was associated with disease recurrence.¹⁶

There remains a need for methods and kits useful to detect metastasis in patients diagnosed with primary colorectal, gastric or esophageal cancer. There remains a need for methods and kits useful to predict the risk of occurrence of relapse among such patients.

### SUMMARY OF THE INVENTION

One aspect of the invention relates to methods of detecting the level of GCC mRNA present in a tissue sample using quantitative (q) RT-PCR. Methods may comprise the steps of: a) isolating RNA from one or more tissue samples obtained from an individual; b) performing quantitative RT-PCR on at least a sample of the RNA using the primers that amplify GCC; c) performing quantitative RT-PCR on at least a sample of the RNA using the primers that amplify a reference marker; and d) estimating by logistic regression analysis of amplification profiles from the quantitative RT-PCR reactions to provide an efficiency-adjusted relative quantification based on parameter estimates from fitted models. In some embodiments the efficiency-adjusted relative quantification is compared to an established cut off.

A related aspect of the invention uses the methods of GCC mRNA levels to determine if a tissue sample contains GCC mRNA indicative of occult metastasis.

Another aspect of the invention provides compositions comprising primers ATTCTAGTGGATCTTTTCAATGACCA (SEQ ID NO:1) and CGTCAGAACAAG-GACATTTTTCAT (SEQ ID NO:2). The composition may further comprise a Taqman probe (FAM-TACTTGGAGGACAATGTCACAG-CCCCTG-TAMRA) (SEQ ID NO:3) and/or CCACACTGTGCCCATCTACG (SEQ ID NO:4) and AGGATCTTCATGAG-GTAGTCAGTCAG (SEQ ID NO:5) and/or Taqman probe (FAM-ATGCCC-X(TAMRA)-CCCCCATGCCATCCTGCGTp) (SEQ ID NO: 6).

A further aspect of the invention relates to kits which comprise a container comprising t primers ATTCTAGTGGATCTTTTCAATGACCA (SEQ ID NO:1) and CGTCAGAACAAG-GACATTTTTCAT (SEQ ID NO:2). and another container comprising Taqman probe (FAM-TACTTGGAGGACAATGTCACAG-CCCCTG-TAMRA) (SEQ ID NO:3). The kits may further comprise primers CACACTGTGCCCATCTACG (SEQ ID NO: 4) and AGGATCTTCATGAG-GTAGTCAGTCAG (SEQ ID NO: 5) and/or Taqman probe (FAM-ATGCCC-X(TAMRA)-CCCCCATGCCATCCTGCGTp) (SEQ ID NO: 6). Kits may optionally include instructions copied to a fixed medium for programming a device to estimate by logistic regression analysis of amplification profiles from quantitative RT-PCR reactions, efficiency-adjusted relative quantifications based on parameter estimates from fitted models.

An additional aspect of the invention provides compositions comprising CCACACTGTGCCCATCTACG (SEQ ID NO:4) and AGGATCTTCATGAG-GTAGTCAGTCAG (SEQ ID NO:5) and optionally (FAM-ATGCCC-X(TAMRA)-CCCCCATGCCATCCTGCGTp) (SEQ ID NO:6).

An aspect of the invention relates to systems for quantifying GCC encoding mRNA by quantitative (q) RT-PCR. Such systems comprise a device programmed to estimate by logistic regression analysis of amplification profiles from quantitative RT-PCR reactions to produce an efficiency-adjusted relative quantification based on parameter estimates from fitted models. The device may also be programmed to compare an efficiency-adjusted relative quantification with established cut off points in order to determine if a sample that was used to produce the efficiency-adjusted relative quantification contained a level of GCC mRNA exceeding a specific threshold.

Another aspect of the invention provides methods of determining the level of GCC mRNA present in a tissue sample using quantitative (q) RT-PCR which comprise the steps of: a) isolating RNA from one or more tissue samples obtained from an individual; and b) performing quantitative RT-PCR on at least a sample of the RNA using the primers that amplify GCC using primers ATTCTAGTGGATCTTTTCAATGACCA (SEQ ID NO:1) and CGTCAGAACAAG-GACATTTTTCAT (SEQ ID NO:2). Some methods further comprise performing quantitative RT-PCR using a Taqman probe (FAM-TACTTGGAGGACAATGTCACAG-CCCCTG-TAMRA) (SEQ ID NO:3).

### DESCRIPTION OF THE FIGURES

FIG. 1. Patient selection for GCC qRT-PCR Analysis.

FIG. 2. Time to recurrence in patients with pN0 Colorectal Cancer Stratified by Occult Lymph Node Metastases. Time to Recurrence in 87 Patients with Stage III pN1 (stage IIIA and stage IIIB) disease is presented for comparison. The likelihood test was used to determine the P value. GUCY2C indicates guanylyl cyclase 2C. pN0 (mol−) indicates lymph nodes negative for GCC. pN0 (mol+) indicates lymph nodes positive for GCC (occult metastasis).

FIG. 3. Multivariate Cox Proportional-Hazards Analysis of Disease Recurrence Risk in Patients with pN0 Colon Cancer Undergoing Molecular Staging. Hazard ratios (black circle) with 95% confidence intervals (horizontal lines) and P values describe interactions between prognostic characteristics (Parameter) and risk of disease recurrence.

FIG. 4. Multivariate Cox Proportional-Hazards Analysis of Disease-Free Survival in Patients with pN0 Colon Cancer Undergoing Molecular Staging. Hazard ratios (black circle) with 95% confidence intervals (horizontal lines) and P values describe interactions between prognostic characteristics (Parameter) and disease-free survival.

FIG. 5. Distribution of GCC mRNA expression in lymph nodes collected from patients with colorectal cancer.

FIG. 6. Time to Recurrence in Colorectal Cancer Patients Stratified by AJCC Stage. Horizontal marks indicate time of last follow-up for individual patients.

FIG. 7. Disease-Free Survival in Colorectal Cancer Patients Stratified by AJCC Stage. Horizontal marks indicate time of last follow-up for individual patients.

FIG. 8. Time to Recurrence in Patients with pN0 Colorectal Cancer Stratified by Number of Lymph Nodes Harboring Occult Metastases. Patients are stratified based on having <3 or >4 lymph nodes harboring occult metastases detected by qRT-PCR. Horizontal marks indicate time of last follow-up for individual patients. Time to recurrence in 87 enrolled patients with stage III N1 (stage IIIA+IIIB) disease is presented for comparison.

FIG. 9. Disease-Free Survival in Patients with pN0 Colorectal Cancer Stratified by Occult Lymph node metastasis. Disease free survival in 87 enrolled patients with stage III pN1 (stage IIIA+IIIB) disease is presented for comparison.

FIG. 10. Disease-Free Survival in Patients with pN0 Colorectal Cancer Stratified by Occult Lymph node metastasis, AJCC stage and anatomical location. Disease free survival for patients with pN0 colorectal cancer and subgroup analysis performed on patients with AJCC Stage 0/1 (A), Stage II (B), Colon (C) and Rectum (D) cancer.

FIG. 11. Time to Recurrence in Patients with pN0 Colorectal Cancer Stratified by Number of Lymph Nodes Harboring Occult Metastases. Patients are stratified based on having ≦3 or ≧4 lymph nodes harboring occult metastases detected by qRT-PCR. Time to recurrence in 87 enrolled patients with stage III N1 (stage IIIA+IIIB) disease is presented for comparison.

### DETAILED DESCRIPTION OF PREFERRED EMBODIMENTS

Methods, kits and systems are provided that can determine relative quantity of GCC mRNA in a sample or series of samples. These methods, kits and systems may be useful to detect metastasis in patients diagnosed with primary colorectal, gastric or esophageal cancer. These methods, kits and systems may be useful to detect metastasis in patients diagnosed with primary colorectal, gastric or esophageal cancer. These methods, kits and systems may be useful to screen individuals for metastatic colorectal, gastric or esophageal cancer. These methods, kits and systems may be useful to predict the risk of occurrence of relapse in patients diagnosed with primary colorectal, gastric or esophageal cancer.

Methods, kits and systems are provided for detecting the level of GCC encoding mRNA present in a sample using quantitative (q) RT-PCR.

In some aspects, the methods comprise the steps of: obtaining one or more tissue samples from an individual; isolating RNA from said sample; and performing quantitative RT-PCR using the primers ATTCTAGTGGATCTTTTCAATGACCA (SEQ ID NO:1) and CGTCAGAACAAG-GACATTTTTCAT (SEQ ID NO:2). In some embodiments, the methods further comprising using a Taqman probe (FAM-TACTTGGAGGACAATGTCACAG-CCCCTG-TAMRA) (SEQ ID NO:3) in the quantitative RT-PCR.

In some aspects of the invention, the methods comprise the steps of: obtaining one or more tissue samples from an individual; isolating RNA from said sample; performing quantitative RT-PCR using the primers that amplify GCC; and performing quantitative RT-PCR using the primers that amplify a reference marker such as beta-actin. In some embodiments the methods comprise performing quantitative RT-PCR using the primers that amplify GCC in which the primers are ATTCTAGTGGATCTTTTCAATGACCA (SEQ ID NO:1) and CGTCAGAACAAG-GACATTTTTCAT (SEQ ID NO:2). In some embodiments, the methods further comprising using a Taqman probe (FAM-TACTTGGAGGACAATGTCACAG-CCCCTG-TAMRA) (SEQ ID NO:3) in the quantitative RT-PCR. In some embodiments, the methods comprise performing quantitative RT-PCR using the primers that amplify beta-actin, in which the primers are CCACACTGTGCCCATCTACG (SEQ ID NO:4) and AGGATCTTCATGAG-GTAGTCAGTCAG (SEQ ID NO:5). In some embodiments, the methods further comprise using a Taqman probe(FAM-ATGCCC-X(TAMRA)-CCCCCATGCCATCCTGCGTp) (SEQ ID NO:6).

In some aspects of the invention, the methods comprise the steps of: obtaining one or more tissue samples from an individual, isolating RNA from said sample, performing quantitative RT-PCR to amplify GCC and a reference marker such as beta-actin, and efficiency adjusting quantitative RT-PCR data based on parameter estimates from fitted models. The efficiency adjusting relative quantity of GCC mRNA may be scored using a predetermined cut off for positive or negative results such as the median efficiency adjusting relative quantity of GCC mRNA in multiple samples from multiple patients. In some embodiments, quantitative RT-PCR to amplify GCC is performed using the primers ATTCTAGTGGATCTTTTCAATGACCA (SEQ ID NO:1) and CGTCAGAACAAG-GACATTTTTCAT (SEQ ID NO:2). In some embodiments, the methods further comprise using a Taqman probe (FAM-TACTTGGAGGACAATGTCACAG-CCCCTG-TAMRA) (SEQ ID NO:3) in the quantitative RT-PCR. In some embodiments, the reference marker is beta-actin and the methods further comprise performing quantitative RT-PCR using the primers that amplify beta-actin using primers CCACACTGTGCCCATCTACG (SEQ ID NO:4) and AGGATCTTCATGAG-GTAGTCAGTCAG (SEQ ID NO:5). In some embodiments, the methods further comprise using a Taqman probe (FAM-ATGCCC-X(TAMRA)-CCCCCATGCCATCCTGCGTp) (SEQ ID NO:6).

In some aspects of the invention, the methods utilize one or more samples from a patient diagnosed with primary colorectal, gastric or esophageal cancer. In some embodiments, the sample is a lymph node sample. In some embodiments, a plurality of samples are used including, for example, 2, 3, 4, 5, 6, 7, 8, 9, 10, 11, 12 or more samples obtained from the patient. In some embodiments, a plurality of lymph node samples are used including, for example, the 2, 3, 4, 5, 6, 7, 8, 9, 10, 11, 12 or more lymph node samples obtained from the patient.

In some aspects of the invention, the data from the methods may be used to determine risk of recurrence.

The present invention provides kits for amplifying GCC-encoding mRNA. The kits may comprise RT-PCR primers ATTCTAGTGGATCTTTTCAATGACCA (SEQ ID NO:1) and CGTCAGAACAAG-GACATTTTTCAT (SEQ ID NO:2). In some embodiments, the kits may further comprise Taqman probe (FAM-TACTTGGAGGACAATGTCACAG-CCCCTG-TAMRA) (SEQ ID NO:3). In some embodiments, the kits may further primers CCACACTGTGCCCATCTACG (SEQ ID NO:4) and AGGATCTTCATGAG-GTAGTCAGTCAG (SEQ ID NO:5). In some embodiments, the kits may further comprise Taqman probe (FAM-ATGCCC-X(TAMRA)-CCCCCATGCCATCCTGCGTp) (SEQ ID NO:6). In some embodiments, the kits may further comprise instructions for programming a device to calculate the relative quantity of GCC mRNA using efficiency adjusting quantitative RT-PCR data based on parameter estimates from fitted models. Such instructions may be copied to a fixed medium. In some embodiments, the kits may further comprise instructions for programming a device to score the results of qPCR samples based upon relative quantity of GCC mRNA using efficiency adjusting quantitative RT-PCR data based on parameter estimates from fitted models. Such scoring may use a predetermined cut off or the median of aggregated data. Such instructions may be fixed to a medium.

The present invention provides compositions for amplifying GCC-encoding mRNA. The compositions may comprise ATTCTAGTGGATCTTTTCAATGACCA (SEQ ID NO:1) and CGTCAGAACAAG-GACATTTTTCAT (SEQ ID NO:2). In some embodiments, the compositions may further comprise (FAM-ATGCCC-X(TAMRA)-CCCCCATGCCATCCTGCGTp) (SEQ ID NO:3).

In some embodiments, the compositions may further comprise CCACACTGTGCCCATCTACG (SEQ ID NO:4) and AGGATCTTCATGAG-GTAGTCAGTCAG (SEQ ID NO:5). In some embodiments, the compositions may further comprise Taqman probe (FAM-ATGCCC-X(TAMRA)-CCCCCATGCCATCCTGCGTp) (SEQ ID NO:6).

The present invention provides systems for quantifying GCC encoding mRNA by quantitative (q) RT-PCR comprising a device programmed to process quantitative RT-PCR data by efficiency adjusting quantitative RT-PCR data based on parameter estimates from fitted models.

The present invention provides systems for determining if a patient has metastatic colorectal, gastric or esophageal cancer by comprising a device programmed to process quantitative RT-PCR data by efficiency adjusting quantitative RT-PCR data based on parameter estimates from fitted models.

The present invention provides for determining risk of recurrence in a patient diagnosed with colorectal, gastric or esophageal cancer comprising a device programmed to process quantitative RT-PCR data by efficiency adjusting quantitative RT-PCR data based on parameter estimates from fitted models.

The methods, kits compositions and systems may also be adapted for determining whether a patient with esophageal dysplasia or otherwise abnormally appearing tissue has Barrett's esophagus. Quantitative RT-PCR amplifying GCC-encoding mRNA may be performed as described herein on esophageal tissue samples to detect GCC mRNA levels and determining whether the results indicate Barrett's esophagus.

One problem associated with the detection of a marker using amplification is the false positives caused by background amplification product. In addition, simple detection assays provide limited information with respect to the degree of marker present. Quantitative amplification such as quantitative PCR overcomes the problems associated with background and provides more information with respect to the degree of target transcript than a simple detection assay.

In addition to the amount of marker present in a sample, quantitative PCR results are affected by the integrity of the sample from the time it is obtained to the time the amplification is performed. Further, the efficiency of the PCR reaction can vary from one sample to another. Thus, when performing quantitative PCR on multiple samples, methods are provided herein to allow for adjusting results to yield relative quantification based results of qPCR of a reference marker such as beta-actin. The GCC qPCR data is adjusted relative to the beta actin qPCR data so that the resulting quantification reflects a relative level of GCC mRNA to reference marker. Accordingly, results can be compared between samples even if a sample has been compromised with respect to degradation or if the reaction performed on a given sample proceeds relatively inefficiently. The relative quantification thereby reduces or eliminates differences in results arising from differences in sample integrity and reaction efficiency among the several samples by producing an output which is normalized with respect to the output from other samples.

By performing quantitative PCR on a reference marker that is present in a sample, such as beta actin, together with performing quantitative PCR on the target marker, such as GCC, the quantitative results of GCC present in a sample can be adjusted and expressed as a relative quantification which corresponds to the number of copies of GCC mRNA as a function of its relationship to the quantity of reference marker. When performing individual quantitative PCR reactions on multiple samples for GCC and a reference marker, the adjustment of results for each sample by logistic regression analyses provides test results which have relative quantification with reduced bias and error. Thus, the results account for the difference in integrity of samples and efficiencies of reactions, yielding relative quantification that more closely reflects the relative amount of amplification target present in the samples.

The reference marker can be any transcript that is known to be present in a sample in an amount within known range. Housekeeping proteins such as beta-actin are useful as reference markers. Amplification of GCC and beta actin transcripts can be performed in a single sample using a multiplex PCR method or a sample can be divided and the reactions can be performed separately. The results of GCC quantification are adjusted based upon the results of the beta actin quantification. By performing beta actin amplifications with GCC amplifications for multiple samples and adjusting the GCC quantification with the beta actin quantification results from the same sample, the resulting output provides a relative quantification of GCC and all results are adjusted to the same standard, reducing or eliminating bias and error from the overall results.

Aspects of the invention relate to methods which include the steps of performing quantitative amplification reactions for GCC and a reference marker such as beta actin and normalizing the GCC results to those for the reference marker to yield a relative quantification of GCC. Each sample is normalized to the reference marker present in that sample to produce relative quantities of GCC with respect to quantities of reference marker. Each relative quantity of GCC determined for each sample can be compared to another other relative quantity of GCC determined for another sample and the comparison reflect the differences in quantification of one sample compared to another, regardless of any differences in sample integrity or reaction efficiencies.

Once relative quantification is determined for multiple samples, the scoring of a sample as positive or negative is achieved by establishing the cut off. One way to establish a cut off is to compile results from a large number of individuals. The median may be calculated and used as the threshold. Those samples in which the relative quantity of GCC are equal to or greater than the median may be scored as positive and those below may be scored as negative. The presence of one positive node can be used to establish an individual as mol+.

As described herein, the quantity of GCC is the relative quantity with respect to the quantity of beta actin rather than an absolute quantification. By calculating relative quantity to a reference marker, the data from all samples is normalized with respect to reference marker and thus to each other. This method removes the variability associated with sample integrity and reaction efficiency that may occur between different samples.

Alternatively, at the time samples are collected, they may be spiked with a known quantity of a reference marker, for example a non-human sequence. Amplification of GCC and the reference maker is performed and quantification results of GCC for may be normalized against the results for the spiked reference marker. It is also envisioned that, the sample may be spiked with a known quantity of a reference marker, for example a non-human sequence, immediately prior to amplification. Amplification of GCC and the reference maker is performed and quantification results of GCC for may be normalized against the results for the spiked reference marker. It is also envisioned that two reference markers may be used, one spiked at the time of collection and one immediately prior to amplification. Spike references may also be used in conjunction with endogenous reference markers.

Systems are provided which include data processing devices which are programmed to calculate relative quantification data by efficiency adjusting quantitative RT-PCR data based on parameter estimates from fitted models. Such devices may be programmed to calculate relative quantities of GCC based upon quantitative results for reference markers such as beta actin. In addition, such devices may be programmed to score results for samples based upon data collected from a plurality of samples. The programming instructions may be provided on a fixed medium which can be used to program a device. A copy of the fixed medium containing the programming instructions may be provided with kits such as those with a container comprising GCC qPCR primers, optionally containers comprising reference marker such as beta actin qPCR primers, optionally positive and/or negative controls and/or instructions for performing the methods.

### Example

The current study prospectively examined the utility of GCC quantitative (q) RT-PCR in patients with pN0 colorectal cancer to identify occult metastases and to define the risk of developing recurrent disease after surgical treatment.

### SUMMARY

Background Approximately 25% of patients with pN0 colorectal cancer develop recurrence after surgery. Guanylyl cyclase C (GCC) is a marker expressed selectively by colorectal tumors. The presence of GCC in histologically negative lymph nodes could indicate the presence of occult metastases and better estimate recurrence risk.

Methods Prospective enrollment of 257 patients with pN0 colorectal cancer at 9 centers provided 2,570 fresh lymph nodes ≧5 mm for histopathology and quantification of GCC mRNA by the reverse transcriptase-polymerase chain reaction (qRT-PCR). Patients were followed for a median of 24 months (range: 2-63) to estimate time to recurrence and disease-free survival.

Results Thirty-two (12.5%) patients had lymph nodes negative by GCC qRT-PCR [pN0(mol−)], and all but two remained free of disease during follow-up (recurrence rate 6.3% [95% CI 0.8-20.8%]). Conversely, 225 (87.5%) patients had lymph nodes positive by GCC qRT-PCR [pN0(mol+)], and 47 (20.9% [15.8-26.8%]) developed recurrent disease (p=0.006). Multivariate analyses revealed that GCC expression in lymph nodes was the most powerful independent prognostic marker. Patients who were pN0(mol+) exhibited an earlier time to recurrence (adjusted hazard ratio 4.42 [1.05-18.53]; p=0.042) and disease-related events associated with reduced disease-free survival (adjusted hazard ratio 3.10 [1.09-8.82]; p=0.034).

Conclusions GCC qRT-PCR positively of histologically negative lymph nodes is independently associated with time to recurrence and disease-free survival in patients with pN0 colorectal cancer. GCC may serve as an indicator of occult lymph node metastases, identifying pN0 patients at high risk for disease recurrence who might benefit from adjuvant chemotherapy.

### Methods

### Study Design

The study was a prospective observational trial. Investigators and clinical personnel were blinded to results of molecular analyses, while laboratory personnel and analysts were blinded to patient and clinical information.

### Patients and Tissues

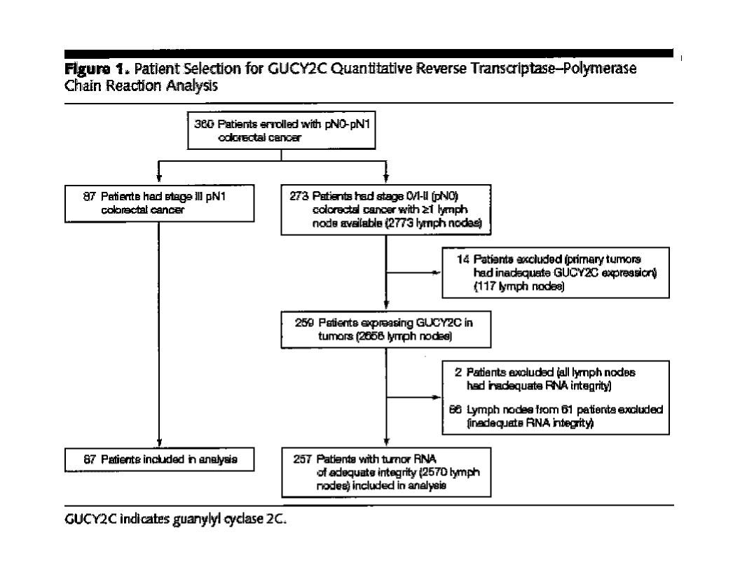

Between March 2002 and June 2007, we enrolled 273 patients with Stage 0 to II pN0 and 87 stage III pN1 colorectal cancer who provided informed consent prior to surgery at one of 7 academic medical centers and 2 community hospitals in the U.S. and Canada (FIG. 1). Patients were ineligible if they had a previous history of cancer, metachronous extra-intestinal cancer, or peri-operative mortality associated with primary resection. For all eligible patients, preoperative and perioperative examinations revealed no evidence of metastatic disease.

Lymph nodes and when available, tumor specimens, were dissected from colon and rectal resections and frozen at −80° C. within one hour to minimize warm ischemia. Half of each resected lymph node was fixed with formalin and embedded in paraffin for histopathological examination. Specimens from stage I and II patients were subjected to molecular analysis if (1) tumor samples, where available, expressed GCC mRNA above background levels in disease-free lymph nodes and (2) at least one lymph node was provided which yielded RNA of sufficient integrity for analysis.¹⁴ Thus, GCC expression in tumors was below background levels in 14 patients who were excluded from further analysis.¹⁴ Moreover, analysis of the 2,656 lymph nodes available from the remaining 259 pN0 patients revealed 86 yielding RNA of insufficient integrity by β-actin qRT-PCR, excluding two additional patients.¹⁴

Overall, the 257 pN0 patients who met eligibility criteria provided 6,699 lymph nodes (range 2-159, median 21 lymph nodes/patient) for histopathologic examination, of which 2,570 nodes (range 1-33, median 8 lymph nodes/patient) were eligible for analysis by qRT-PCR. The greater number of lymph nodes available for histopathology versus molecular analysis from pN0 patients includes those collected after formalin fixation or nodes <5 mm in diameter, smaller than the limit of bisection.

Disease status, obtained in routine follow-up by treating physicians, was provided for all patients through December 2007.

### RNA Isolation

RNA was extracted from tissues by a modification of the acid guanidinium thiocyanate-phenol-chloroform extraction method.^{16, 17} Briefly, individual tissues were pulverized in 1.0 mL Tri-Reagent (Molecular Research Center, Cincinnati, Ohio) with 12-14 sterile 2.5 mm zirconium beads in a bead mill (Biospec, Bartlesville, Okla.) for 1-2 min. Phase separation was performed with 0.1 mL bichloropropane, and the aqueous phase re-extracted with 0.5 mL chloroform. RNA was precipitated with 50% isopropanol and washed with 70% ethanol. Air-dried RNA was dissolved in water, concentration determined by spectrophotometry, and stored at −80° C.

### RT-PCR

GCC mRNA was quantified by RT-PCR employing an established analytically validated assay.¹⁴ The EZ RT-PCR kit (Applied Biosystems, Foster City, Calif.) was employed to amplify GCC mRNA from total RNA in a 50 μL reaction. Optical strip-tubes were used for all reactions, which were conducted in an ABI 7000 Sequence Detection System (Applied Biosystems, Foster City, Calif.). In addition to the kit components [50 mM Bicine (pH 8.2), 115 mM KOAc, 10 μM EDTA, 60 nM ROX, 8% glycerol, 3 mM Mg(OAc)₂, 300 μM each dATP, dCTP, and dGTP, 600 μM dUTP, 0.5 U uracil N-glycosylase, and 5 U rTth DNA polymerase], the reaction master mix contained 900 nM each of forward (ATTCTAGTGGATCTTTTCAATGACCA—SEQ ID NO:1) and reverse primers (CGTCAGAACAAG-GACATTTTTCAT—SEQ ID NO:2), 200 nM Taqman probe (FAM-TACTTGGAGGACAATGTCACAG-CCCCTG-TAMRA), and 1 μg RNA template. The housekeeping gene β-actin was amplified employing similar conditions except that forward (CCACACTGTGCCCATCTACG) and reverse (AGGATCTTCATGAG-GTAGTCAGTCAG) primers were 300 nM each, while the Taqman probe (FAM-ATGCCC-X(TAMRA)-CCCCCATGCCATCCTGCGTp) was 200 nM. The thermocycler program employed for RT included: 50°×2 min, 60°×30 min, 95°×5 min; and for PCR: 45 cycles of 94°×20 sec, 62°×1 min. Reactions were performed at least in duplicate and results averaged.

### Statistical Analysis

To have at least 80% power to detect a hazard ratio of 1.6, based on a 2-sided test P≦0.05, 225 patients with pN0 colorectal cancer were required. GCC and 3-actin mRNA were estimated by logistic regression analyses of amplification profiles from individual RT-PCR reactions, providing an efficiency-adjusted relative quantification based on parameter estimates from the fitted models which reduces bias and error (see Relative Quantification of GCC Expression by qRT-PCR, below, for further details).¹⁹ The distribution of relative GCC expression for each lymph node was quantified and the overall median computed.

A priori, nodes in which relative GCC mRNA was greater than or equal to the median were considered positive while those less than the median were considered negative, in the absence of established methodologies to define optimal cutpoints for molecular markers from multiple measurements for individual patients. Patients were considered pN0(mol+) if 1 or more nodes were positive.

The primary clinical endpoint was time to recurrence, measured from the date of surgery to the time of the last follow-up, recurrence event or death.²⁰ Disease-free survival, defined as time from surgery to any event regardless of cause, was a secondary clinical outcome.²⁰ Confidence intervals for raw survival rates were computed by the method of Clopper-Pearson.²¹ Survival distributions for patients with and without occult metastases were compared employing the likelihood ratio test. While Kaplan-Meier plots display censored survival at 36 months to ensure availability of at least 20% of patients at all time points, analyses incorporated all events up to date of last follow-up.²² The association of pN0(mol+) with categorical patient characteristics was quantified using chi-square tests or the Fisher's exact test in cases of small sample sizes. Simultaneous prognostic effects of different parameters were estimated employing Cox regression analysis. Established prognostic variables in the Cox model for recurrence included T stage; chemotherapy; tumor size, location, and differentiation; lymphovascular invasion; and pN0 molecular status.²³ The multivariable model for each outcome included all of the established prognostic measures regardless of significance in order to establish the additional independent prognostic effect of molecular status. All tests were two-sided, and p<0.05 was considered statistically significant.

### Relative Quantification of GCC Expression by QRT-PCR

GCC and β-actin expression was estimated by logistic regression analysis of amplification profiles from individual RT-PCR reactions, providing an efficiency-adjusted relative quantification based on parameter estimates from the fitted models which reduces bias and error.¹⁹ In the re-parameterized logistic model:

$$F ( x ) = L + \frac{U - L}{1 + {e}^{m} {A}^{- x}} , \qquad ( 1 )$$

where L and U=L+PK are lower and upper asymptotes, respectively, A is the maximum amplification rate, and m=ln(K/N(0)-1), where) N(0) is the number of starting templates in the reaction, m may be used to compute the log-ratio expression of a target gene normalized to a reference gene. For real RT-PCR reactions, N(0) is less than K by orders of magnitude, and therefore

m=ln(K/N(0)−1)≈ ln(K)−ln(N(0)),

where K may either be the same for target and reference reactions, or, at least, the same constant for all target reactions and another constant for all reference reactions. Hence, up to a constant shift, common for all reactions, the log-ratio of a target normalized to a reference may be computed as

ln R_{T/R}=ln N_{T}(0)−ln N_{R}(0)>>m_{R}−m_{T} (2)

where m_{T} and m_{R} are m parameters in model (1) for target and reference gene reactions, respectively.

If one considers the nonlinear model for fluorescence Fᵢ at cycle xᵢ:

$${F}_{i} = L + \frac{U - L}{1 + {e}^{m} {A}^{- {x}_{i}}} + {ɛ}_{i} , \qquad ( 3 )$$

where εᵢ˜i.i.d. N(0, σ) represent measurement errors. Fitting (3) using standard non-linear regression methods provides the estimates \hat{m}_{T} and \hat{m}_{R} and their standard errors, se(\hat{m}_{T}) and se(\hat{m}_{R}) for each target and reference gene reaction. Then the log-ratio of a target normalized to a reference is estimated as:

$$= {\hat{m}}_{R} - {\hat{m}}_{T} \qquad ( 4 )$$

and the standard error of

is computed as

$$se [ \text{?} ] = \sqrt{{[ se ( \text{?} ) ]}^{2} + {[ se ( \text{?} ) ]}^{2}} . \\ se [ ] = \sqrt{{[ se ( {\hat{m}}_{T} ) ]}^{2} + {[ se ( {\hat{m}}_{R} ) ]}^{2}} . \\ \text{?} \text{indicates text missing or illegible when filed} \qquad ( 5 )$$

Here, the qRT-PCR fluorescence profile for GCC and beta-actin for each lymph node was exported to Excel data files, imported to SAS, and fit using model (3) with the Nonlin procedure. Parameter estimates, measures of goodness of fit and convergence status were recorded for each reaction and used for further analysis. Each lymph node was run for each gene in duplicate, and averages for each node computed. In that context, for n_{T} replicates of target and n_{R} replicates of reference RT-PCR reactions for the same biological sample, let \hat{m}_{Ti} i=1, . . . , n_{T} and \hat{m}_{Ri}, i=1, . . . , n_{R} be non-linear regression estimates of parameter m from model (3) with the corresponding estimated standard errors se(\hat{m}_{Ti}) i=1, . . . , n_{T} and se(\hat{m}_{Ri}^{|}) i=1, . . . , n_{R}.

Denote

$${\overline{m}}_{T} = \frac{1}{{n}_{T}} {\sum }_{i = 1}^{{n}_{T}} {\hat{m}}_{Ti}$$

$${\overline{m}}_{R} = \frac{1}{{n}_{R}} {\sum }_{i = 1}^{{n}_{T}} {\hat{m}}_{Ri} .$$

For the same biological sample, replicates are considered independent, conditional on the random effect of a sample or an individual. The log-ratio and its standard error may be computed as:

$$= {\overline{m}}_{R} - {\overline{m}}_{T} \\ se [ ] = \sqrt{\frac{1}{{n}_{T}^{2}} {\sum }_{i = 1}^{{n}_{T}} {[ se ( {\hat{m}}_{Ti} ) ]}^{2} + \frac{1}{{n}_{R}^{2}} {\sum }_{i = 1}^{{n}_{R}} {[ se ( {\hat{m}}_{Ri} ) ]}^{2}} . \qquad ( 6 )$$

Here, relative GCC expression was computed for each lymph node for each patient using this approach. For any reaction where the logistic model did not converge, or did not exhibit goodness of fit measuring ≧80%, or if the amplification constant, A in model (1), was not ≧1.5, the fluorescence isotherms were individually reviewed by two members of the research team. In all cases where this occurred for GCC, reactions did not amplify, implying zero or low expression of the gene. For the same lymph node, if β-actin expression was >2000 copies, representing the 5^{th} percentile of beta-actin expression¹⁴, then it was presumed the sample had viable RNA, and GCC expression was set to the lowest measured value of GCC expression. Nodes where β-actin expression <2000 copies were eliminated from further analysis.

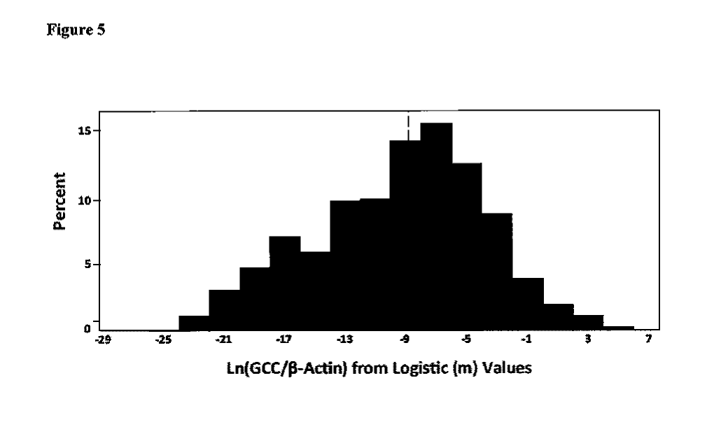

The distribution of relative GCC expression for each lymph node was quantified, averaged over replicates, and the median computed. As a conservative approach for this analysis, nodes where relative GCC expression was ≧median were considered positive, while those <median were considered negative. (FIG. 5) Median expression was specifically selected a priori as the threshold because it maximizes the probability of identifying patients harboring occult metastases in context of variable collections of lymph nodes from individual patients. In this analysis, median expression was estimated as about 173 copies of GCC mRNA, closely approximating that obtained in earlier studies (about 200 copies) employing different samples and analytic approaches, reinforcing the validity of the techniques. Employing this threshold provides a sensitivity and specificity of 93% and 78%, respectively, when applied to the validation cohort of true positive and negative lymph nodes defined previously. Lymph nodes for each patient were then summarized to compute the number of positive lymph nodes. For Kaplan-Meier and Cox analyses, this was categorized as zero nodes positive=pN0[mol−] or ≧1 nodes positive=pN0[mol+]. In an additional subgroup where >12 lymph nodes were available for each patient, the categories 0 to 3 lymph nodes positive and ≧4 lymph nodes positive were applied, which are comparable to those employed in histopathological staging and risk stratification in colorectal cancer.^{3, 23}.

### Results

### Patient Characteristics

The 257 pN0 patients whose lymph nodes were subjected to qRT-PCR had a mean age of 68 years at diagnosis and 44.8% were female (Table 1). Clinicopathologic features, including depth of tumor penetration (T1/2, T3, T4), and tumor anatomical location (right, left, sigmoid colon) were similar to national experience^{3, 4, 23} Patients with colon cancer represented 87.4%, while those with rectal tumors were 13.6%.

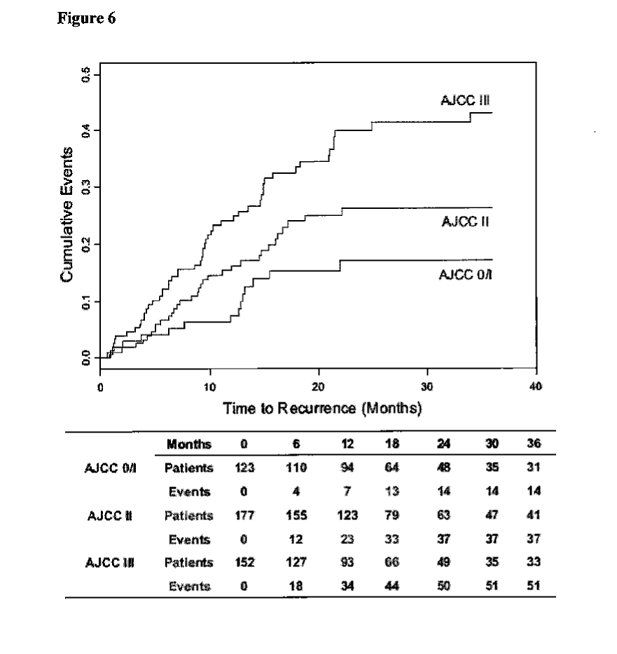

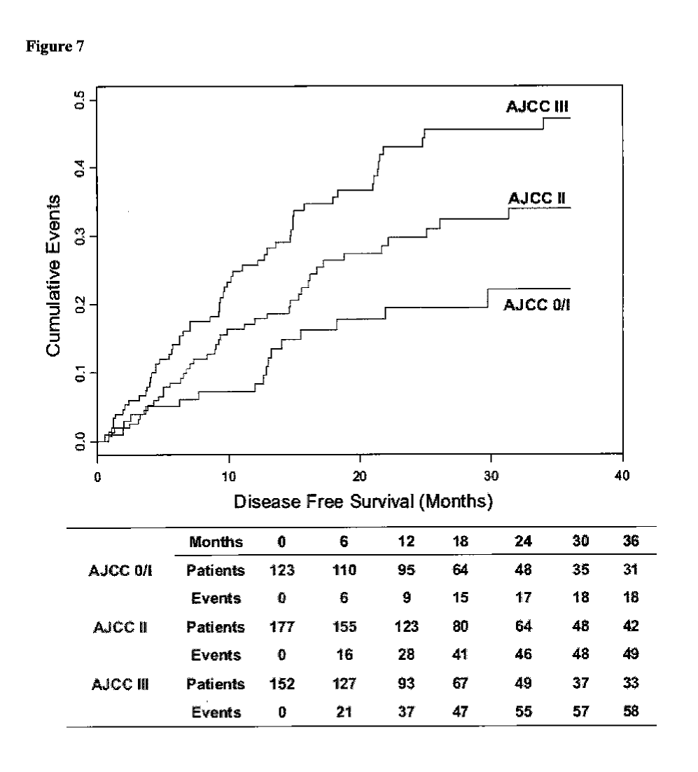

There were no statistically significant differences in the baseline characteristics of patients included vs. those excluded from qRT-PCR analysis and in those with and without occult metastases, with the exception of tumor grade (Table 1). Patients exhibited the well-established direct relationships between time to recurrence, disease-free survival and stage (FIGS. 6, 7).^{3, 4, 23} Twenty-two percent of patients with pN0 and 71.3% with stage III, colon cancer received adjuvant 5-fluorouracil-based chemotherapy.

### Occult Metastases and Disease Recurrence

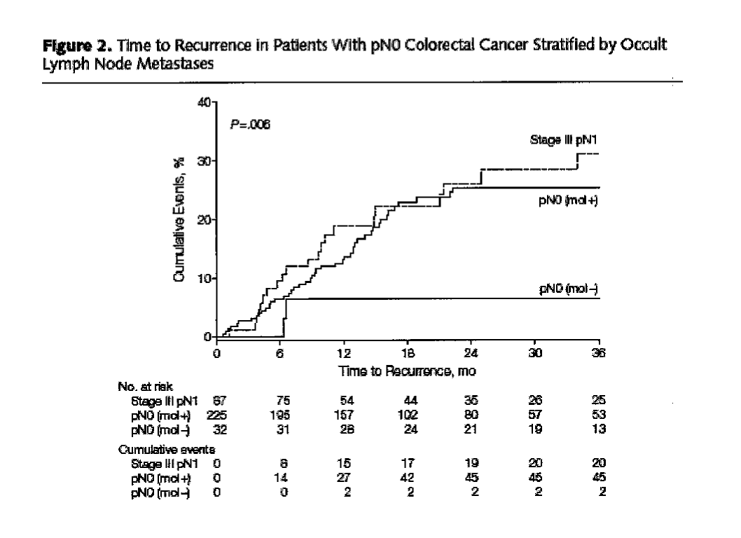

GCC expression, presumably indicating the presence of occult metastases, was detected in at least one lymph node from 225 (87.5%) patients with pN0 colorectal cancer. With a median follow-up of 24 months (range, 1.8 to 62.7) for patients with pN0(mol+) and 35.9 months (range, 2.5-62.1) for patients with pN0(mol−), 20.9% (CI, 15.8-26.8%) of patients with, but only 6.3% (CI, 0.8-20.8%) without, occult metastases developed recurrent disease. Reflecting the established insensitivity of staging employing inadequate lymph node sampling^{3, 23-25}, both GCC-negative patients who developed recurrent disease provided ≦2 lymph nodes for analysis by qRT-PCR. Patients who were pN0(mol+) exhibited a cumulative incidence of recurrence that was more than 3-fold greater than pN0(mol−) patients (FIG. 2; p=0.006).

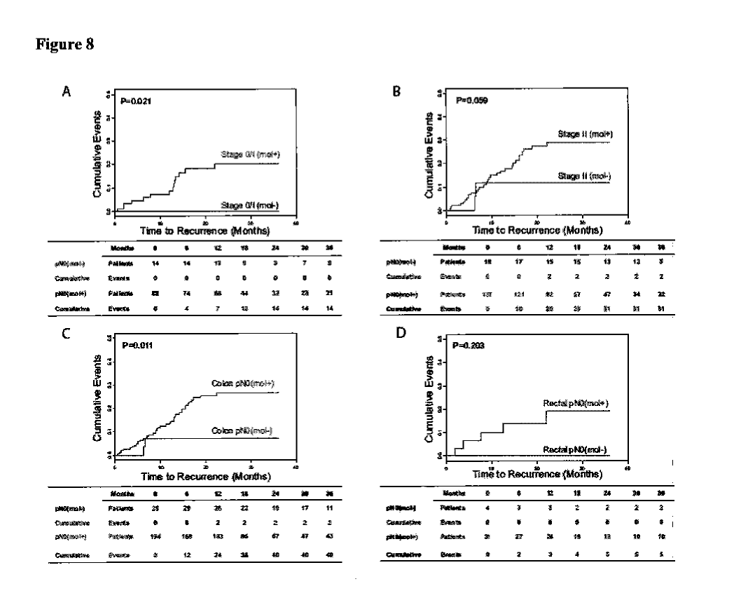

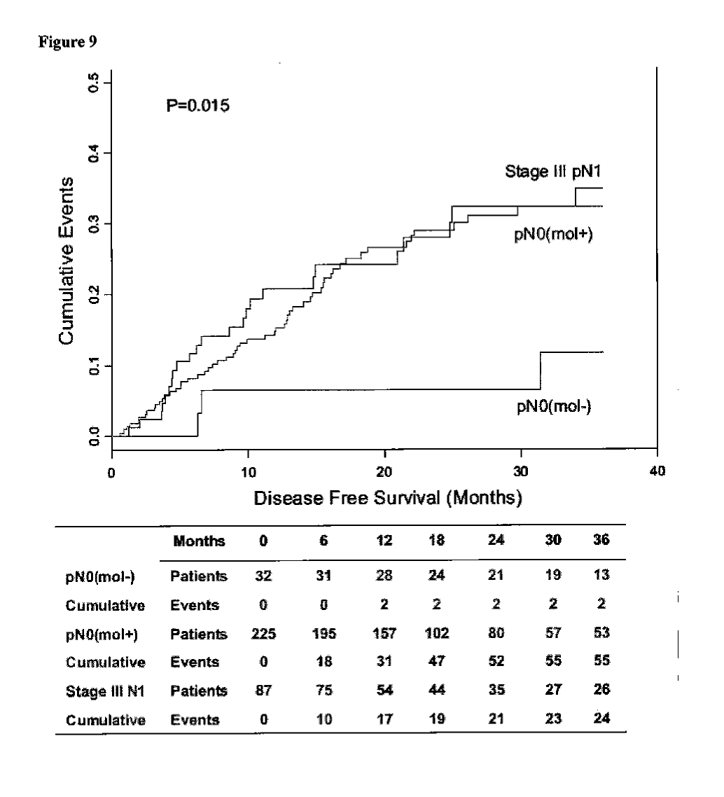

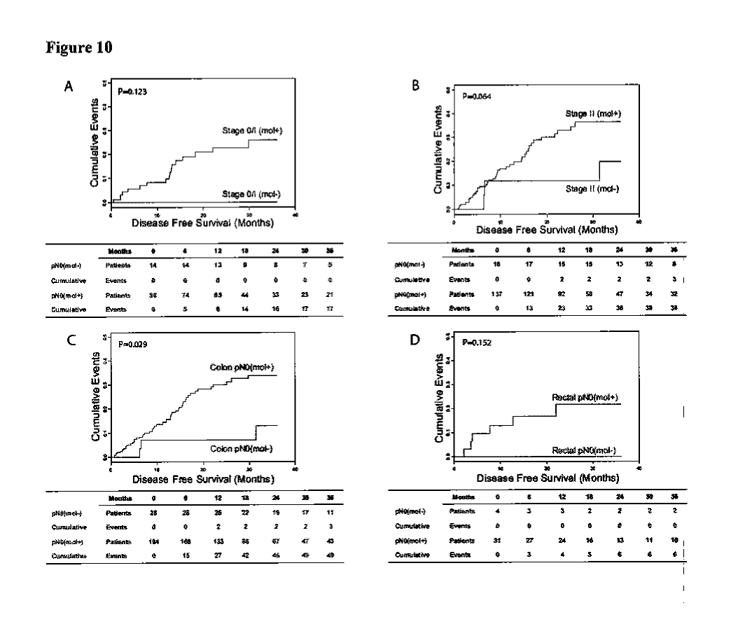

Subgroup analyses revealed that GCC positively conferred significantly worse prognosis among patients with MCC stage I and II and those with colon cancer (FIG. 8). Moreover, GCC positive lymph nodes were associated with reduced disease-free survival (FIG. 9). Patients who were pN0(mol+) exhibited cumulative disease events that were more than 2-fold greater than pN0(mol−) patients (FIG. 9, p=0.015). Like time to recurrence, subgroup analyses suggest that occult metastases were associated with reduced disease-free survival in patients with tumors of different stages and locations (FIG. 10). Time to recurrence (FIG. 2), disease-free survival (FIG. 9), and the cumulative incidence of recurrence and disease events in pN0(mol+) patients were comparable to that of patients with stage III N1 (stage IIIA+IIIB) disease, all of whom have histopathologically-detectable metastases in regional lymph nodes.

### GCC Positively as a Prognostic Variable

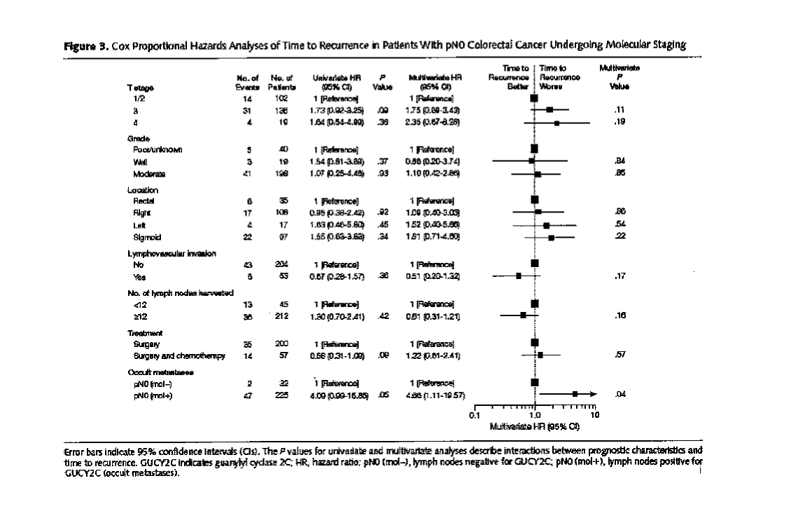

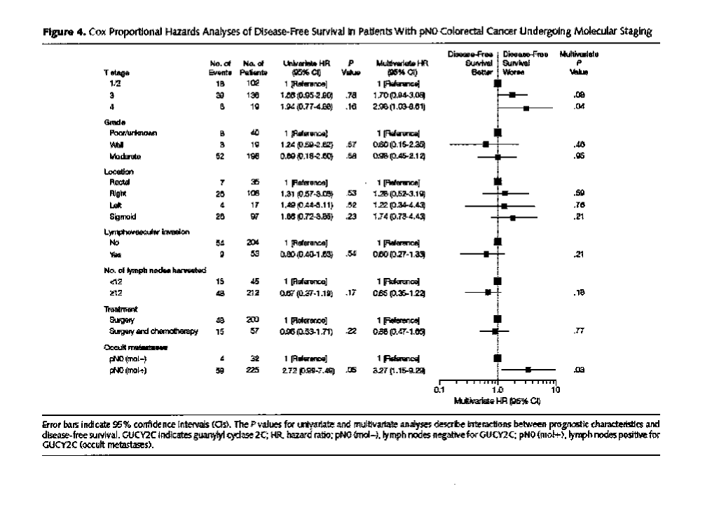

Univariate (Tables 2, 3) and multivariate analyses employing Cox proportional-hazards models (FIGS. 3 and 4) revealed that grade, tumor location, and lymphatic or vascular invasion contributed little as prognostic factors in our cohort of patients with pN0 colorectal cancer. T stage was a weak prognostic variable, reflecting the disproportionate number of T3 (52.9%), compared to T4 (7.4%), tumors in the pN0 cohort and the established relationship between tumor size, depth of penetration and prognosis.^{3, 4, 9, 23}. The presence of GCC positively provided the greatest independent prognostic information. Patients who were pN0(mol+) exhibited an increased hazard of earlier time to recurrence (absolute event rates: pN0(mol−), 6.3%; pN0 (mol+), 20.9%; adjusted hazard ratio 4.66 [95% CI, 1.11-19.57]; p=0.04; FIG. 3), and disease-related events associated with reduced disease-free survival (absolute event rates: pN0(mol−), 12.5%; pN0 (mol+), 26.2%; adjusted hazard ratio 3.27 [95% CI, 1.15-9.29; p=0.03; FIG. 4).

### Discussion

A near-universal principle of cancer staging recognizes the established relationship between regional lymph node metastases and prognostic risk.^{4, 23} In colon and rectal cancer, lymph node metastasis is the single most important prognostic characteristic, representing pathologic evidence of dissemination of tumor cells beyond their primary location. Clinically, approximately 50% of stage III patients will experience disease recurrence.^{1, 2, 4, 9, 23-26} Because up to 25% of pN0 patients, i.e. patients without histological evidence of nodal involvement, also experience recurrent disease, it is presumed that many such patients harbor occult metastases not identified by histopathology at the time of primary resection.^{1, 2} Under staging by conventional methods reflects the combination of insufficient tissue sampling for review, the analysis of small volumes of individual lymph node tissue missing metastatic tumor cells²⁷, and the sensitivity of histopathology, which reliably detects only 1 cancer cell in 200 normal cells²⁸. Molecular staging could overcome limitations in the detection of occult lymph node metastases by incorporating all available tissue into analyses, and increasing detection sensitivity by employing quantifiable disease-specific molecular markers^{1, 11} which nominally identify a single cancer cell in 1 million normal cells²⁹.

In this study, prospective detection of occult metastases by GCC qRT-PCR appeared to be an independent prognostic marker of risk. Molecular staging revealed that about 13% of patients with pN0 colorectal cancer were free of tumor cells, while about 87% had GCC results that suggested occult metastases. Even in the context of shorter follow-up, which could introduce a bias against the utility of GUCY2C in this setting, patients who were pN0(mol+) exhibited a significantly greater risk of earlier disease recurrence and reduced disease-free survival, the primary and secondary outcomes of the study, compared with patients with pN0 (mol−). While enrollment was sufficient to satisfy requirements for these outcomes, the 95% CIs around estimates in multivariate analyses were broad. Future studies with greater numbers of patients should provide more precise estimates of the prognostic utility of GCC quantitative RT-PCR.

Although a high proportion of pN0 patients have GCC positively, indicating occult metastases, most pN0 patients will not recur.^{3, 23} As noted above, not all stage III patients, who have histopathologically-detectable lymph node metastases ultimately develop recurrent disease.^{3, 23} Reconciliation of this apparent inconsistency relies on the recognition that the presence of nodal metastases, regardless of methods used to detect them, does not assure recurrence, but it does indicate its risk. In support of this concept, our study suggests nearly recurrence rates for pN0(mol+) patients with occult metastases that are nearly identical to these for stage III pN1 patients³, the lowest stage in which all patients have histopathologically-detectable metastases (see FIGS. 2, 9).^{3, 4}

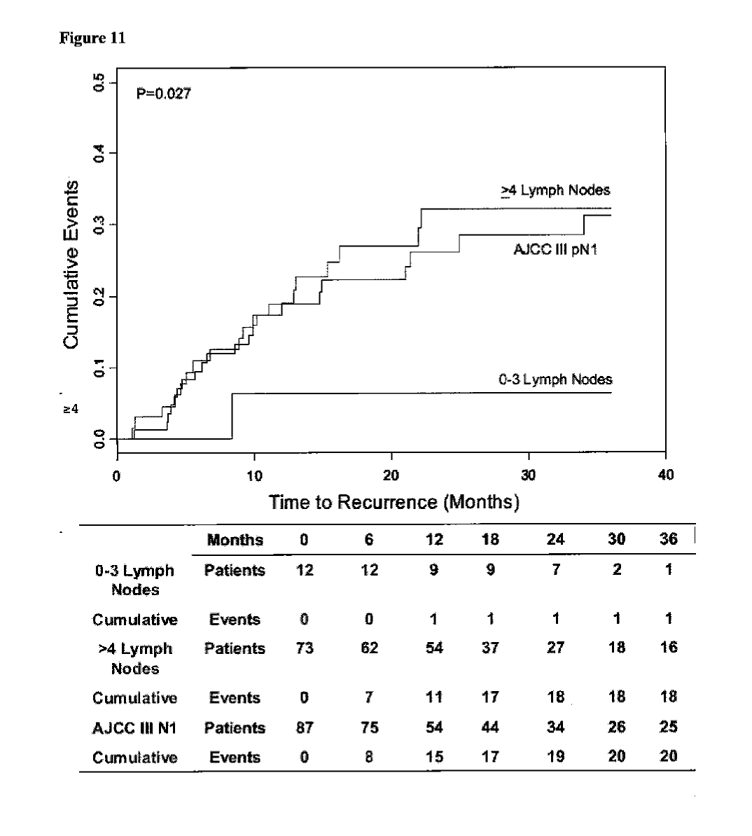

There is also an established relationship between prognostic risk and burden of disease, quantified as the number of lymph nodes harboring tumor cells by histopathology. Assuming there are adequate numbers of lymph nodes to review, stage III patients with ≧4 involved lymph nodes exhibit a recurrence rate that is approximately 50-100% greater than those with ≦3 involved nodes^{3, 23} Estimates of tumor burden and staging precision are intimately related to the number of lymph nodes analyzed. Histopathologic review of ≧12 lymph nodes establishes a diagnosis of pN0 with optimum accuracy^{3, 23-25}, while staging imprecision contributes to less predictable patient outcomes when ≦2 lymph nodes are analyzed.^{3, 23-25} One limitation of the present study is the variable number of lymph nodes available for molecular staging from individual patients, reflecting the requirement for fresh dissection of surgical specimens. Additionally, lymph nodes <5 mm were excluded from molecular analyses, reflecting size limits for tissue bisection, although they are a particularly rich source of tumor metastases.^{30, 31} These considerations suggest that the precision of staging by molecular analyses could benefit from optimum lymph node sampling to incorporate tumor burden into prognostic risk stratification.^{1, 2, 26} An analysis of the subset of pN0 patients providing 12 lymph nodes for GCC qRT-PCR applying standard American Joint Committee on Cancer definitions for stage N1 and N2^{3, 23}, revealed that those with 0-3 involved nodes exhibited a prognostic risk similar to pN0(mol−) patients (5.9% v 8.3%, respectively; FIG. 11). Conversely, those with ≧4 involved nodes exhibited a risk (≦3 versus ≧4, p=0.03) identical to patients with stage III N1 disease (FIG. 8). Improved prognostic risk stratification by integrating detection of occult metastases and estimates of tumor burden underscores the essential importance of adequate lymph node sampling for optimal molecular^{1, 2, 26}, as well as histopathological^{3, 23-25}, staging of patients with colorectal cancer.

The number of involved lymph nodes notwithstanding, there is an evolving relationship between the volume of cancer cells in individual nodes, disease burden, and prognostic risk.^{3, 27} Metastases ≧0.2 mm are associated with increased disease recurrence.³ However, the relationship between individual tumor cells or nests smaller than 0.2 mm and prognostic risk remains undefined.³ The emergence of quantitative RT-PCR provides an unprecedented opportunity for cancer cell enumeration in tissues. The superior sensitivity of RT-PCR²⁹, with its optimal tissue sampling and capacity for single cell discrimination, could identify occult cancer cells in lymph nodes below the threshold of prognostic risk³, limiting the specificity of molecular staging. In that context, the current study was not designed to identify the quantitative threshold defining risk. Indeed, one limitation of this study was the requirement to define a priori the diagnostic limit of GCC. In future studies, it will be essential to more precisely define the quantitative relationship between marker expression and disease risk that incorporates tumor burden to optimize prognostic sensitivity and specificity.

The presence of tumor cells in regional lymph nodes also directs therapy in patients with colon cancer. While adjuvant chemotherapy provides a survival benefit to patients with stage III disease, its utility in patients with pN0 colon cancer remains uncertain, with marginal survival benefits in stage II patients in some, but not all, clinical trials.^{3, 5-9, 23, 32, 33} This uncertainty of treatment benefit in stage II patients is echoed in the dynamic evolution of treatment guidelines, in which adjuvant therapy has become discretionary in stage II patients with clinicopathologic features of poor prognostic risk, including T4 stage, intestinal obstruction, and intestinal perforation.^{9, 32, 34, 35} The heterogeneous responses to therapy in pN0 patients may reflect, in part, heterogeneity with respect to occult nodal metastases. Moreover, standard of care includes adjuvant chemotherapy for stage III N1 patients, a cohort with a recurrence rate identical to pN0(mol+) patients (see FIGS. 2, 9). These considerations highlight the importance of advancing beyond the present study to refine the prognostic specificity of molecular staging using GUCY2C quantitative RT-PCR to more precisely stratify risk in patients with pN0 colorectal cancer and better inform the use of adjuvant chemotherapy.

Molecular staging represents one component of a comprehensive diagnostic, prognostic and predictive paradigm to personalize management strategies for individual patients.^{36, 37} It provides adjunctive clinicopathological information that supplements, but does not replace, complimentary anatomical, microscopic, and morphological staging modalities. Beyond enhancing these current approaches, molecular staging offers a unique opportunity to prioritize future complex resource-intensive analyses of primary tumors that will optimize patient management. In that context, analyses of primary tumors to define mutations, gene expression and epigenetic profiles, and proteomic signatures to stratify risk, predict responses to chemotherapy, and personalize interventions, may best be applied to pN0(mol+), rather than pN0(mol−), patients.³⁸⁻⁴² These considerations underscore the present and future importance of integrating molecular approaches incorporating specific markers of disease, like GCC, and powerful detection methods like qRT-PCR, into analytical paradigms directing the management of patients with colorectal cancer.

### REFERENCES

1. Iddings D, Ahmad A, Elashoff D, Bilchik A. The prognostic effect, of micrometastases in previously staged lymph node negative (N0) colorectal carcinoma: a meta-analysis. Ann Surg Oncol 2006; 13:1386-92.
2. Nicastri D G, Doucette J T, Godfrey T E, Hughes S J. Is occult lymph node disease in colorectal cancer patients clinically significant? A review of the relevant literature. J Mol Diagn 2007; 9:563-71.
3. Compton C C, Greene F L. The staging of colorectal cancer: 2004 and beyond. CA Cancer J Clin 2004; 54:295-308.
4. Jemal A, Siegel R, Ward E, Murray T, Xu J, Thun M J. Cancer statistics, 2007. CA Cancer J Clin 2007; 57:43-66.
5. Andre T, Boni C, Mounedji-Boudiaf L, et al. Oxaliplatin, fluorouracil, and leucovorin as adjuvant treatment for colon cancer. N Engl J Med 2004; 350:2343-51.
6. Mamounas E, Wieand S, Wolmark N, et al. Comparative efficacy of adjuvant chemotherapy in patients with Dukes' B versus Dukes' C colon cancer: results from four National Surgical Adjuvant Breast and Bowel Project adjuvant studies (C-01, C-02, C-03, and C-04). J Clin Oncol 1999; 17:1349-55.
7. Meyerhardt J A, Mayer R J. Systemic therapy for colorectal cancer. N Engl J Med 2005; 352:476-87.
8. Quasar Collaborative G, Gray R, Barnwell J, et al. Adjuvant chemotherapy versus observation in patients with colorectal cancer: a randomised study. Lancet 2007; 370:2020-9.
9. Wolpin B M, Meyerhardt J A, Mamon H J, Mayer RJ. Adjuvant treatment of colorectal cancer. CA Cancer J Clin 2007; 57:168-85.
10. Waldman S A, Terzic A. Therapeutic burden In cancer revealed by molecular staging. Biomarkers in Medicine 2008; In press.
11. Li P, Schulz S, Bombonati A, et al. Guanylyl cyclase C suppresses intestinal tumorigenesis by restricting proliferation and maintaining genomic integrity. Gastroenterology 2007; 133:599-607.
12. Pitari G M, Li P, Lin J E, et al. The paracrine hormone hypothesis of colorectal cancer. Clin Pharmacol Ther 2007; 82:441-7.
13. Birbe R, Palazzo J P, Walters R, Weinberg D, Schulz S, Waldman SA. Guanylyl cyclase C is a marker of intestinal metaplasia, dysplasia, and adenocarcinoma of the gastrointestinal tract. Hum Pathol 2005; 36:170-9.
14. Schulz 5, Hyslop T, Haaf J, et al. A validated quantitative assay to detect occult micrometastases by reverse transcriptase-polymerase chain reaction of guanylyl cyclase C in patients with colorectal cancer. Clin Cancer Res 2006; 12:4545-52.
15. Witek M E, Nielsen K, Walters R, et al. The putative tumor suppressor Cdx2 is overexpressed by human colorectal adenocarcinomas. Clin Cancer Res 2005; 11:8549-56.
16. Cagir B, Gelmann A, Park J, et al. Guanylyl cyclase C messenger RNA is a biomarker for recurrent stage II colorectal cancer. Ann Intern Med 1999; 131:805-12.
17. Carrithers S L, Barber M T, Biswas S, et al. Guanylyl cyclase C is a selective marker for metastatic colorectal tumors in human extraintestinal tissues. Proc Natl Acad Sci U S A 1996; 93:14827-32.
18. Frick G S, Pitari G M, Weinberg D S, Hyslop T, Schulz S, Waldman S A. Guanylyl cyclase C: a molecular marker for staging and postoperative surveillance of patients with colorectal cancer. Expert Rev Mol Diagn 2005; 5:701-13.
19. Chervoneva I, Li Y, Iglewicz B, Waldman S, Hyslop T. Relative quantification based on logistic models for individual polymerase chain reactions. Stat Med 2007; 26:5596-611.
20. Punt C J, Buyse M, Kohne C H, et al. Endpoints in adjuvant treatment trials: a systematic review of the literature in colon cancer and proposed definitions for future trials. J Natl Cancer Inst 2007; 99:998-1003.
21. Newcombe R G. Two-sided confidence intervals for the single proportion: comparison of seven methods. Stat Med 1998; 17:857-72.
22. Pocock S J, Clayton T C, Altman D G. Survival plots of time-to-event outcomes in clinical trials: good practice and pitfalls. Lancet 2002; 359:1686-9.
23. Greene F L. AJCC Cancer Staging Manual. 6th ed. New York: Springer; 2002.
24. Le Voyer T E, Sigurdson E R, Hanlon A L, et al. Colon cancer survival is associated with increasing number of lymph nodes analyzed: a secondary survey of intergroup trial INT-0089. J Clin Oncol 2003; 21:2912-9.
25. Swanson R S, Compton C C, Stewart A K, Bland K I. The prognosis of T3N0 colon cancer is dependent on the number of lymph nodes examined. Ann Surg Oncol 2003; 10:65-71.
26. Bilchik A J, Hoon D S, Saha S, et al. Prognostic impact of micrometastases in colon cancer: interim results of a prospective multicenter trial. Aim Surg 2007; 246:568-75; discussion 75-7.
27. Hitchcock C L, Sampsel J., Young D C, Martin E W, Jr., Arnold M W. Limitations with light microscopy in the detection of colorectal cancer cells. Dis Colon Rectum 1999; 42:1046-52.
28. Ratto C, Sofo L, Ippoliti M, et al. Accurate lymph-node detection in colorectal specimens resected for cancer is of prognostic significance. Dis Colon Rectum 1999; 42:143-54; discussion 54-8.
29. Nolan T, Hands R E, Bustin S A. Quantification of mRNA using real-time RT-PCR. Nat Protoc 2006; 1:1559-82.
30. Brown H G, Luckasevic T M, Medich D S, Celebrezze J P, Jones S M. Efficacy of manual dissection of lymph nodes in colon cancer resections. Mod Pathol 2004; 17:402-6.
31. Herrera-Ornelas L, Justiniano J, Castillo N, Petal N J, Stulc J P, Mittelman A. Metastases in small lymph nodes from colon cancer. Arch Surg 1987; 122:1253-6.
32. Benson A B, 3rd, Schrag D, Somerfield M R, et al. American Society of Clinical Oncology recommendations on adjuvant chemotherapy for stage II colon cancer. J Clin Oncol 2004; 22:3408-19.
33. Gill S, Loprinzi C L, Sargent D J, et al. Pooled analysis of fluorouracil-based adjuvant therapy for stage II and III colon cancer: who benefits and by how much? J Clin Oncol 2004; 22:1797-806.
34. Figueredo A, Charette M L, Maroun J, Brouwers M C, Zuraw L. Adjuvant therapy for stage II colon cancer: a systematic review from the Cancer Care Ontario Program in evidence-based care's gastrointestinal cancer disease site group. J Clin Oncol 2004; 22:3395-407.
35. Winn R, McClure J. The NCCN clinical practice guidelines in oncology. J Natl Comprehensive Cancer Network 2005; 1.
36. Waldman S A, Christensen N B, Moore J E, Terzic A. Clinical pharmacology: the science of therapeutics. Clin Pharmacol Ther 2007; 81:3-6.
37. Waldman S A, Terzic M R, Terzic A. Molecular medicine hones therapeutic arts to science. Clin Pharmacol Ther 2007; 82:343-7.
38. Croner R S, Peters A, Brueckl W M, et al. Microarray versus conventional prediction of lymph node metastasis in colorectal carcinoma. Cancer 2005; 104:395-404.
39. Frigola J, Song J, Stirzaker C, Hinsheiwood R A, Peinado M A, Clark S J. Epigenetic remodeling in colorectal cancer results in coordinate gene suppression across an entire chromosome band. Nat Genet 2006; 38:540-9.
40. Jen J, Kim H, Piantadosi S, et al. Allelic loss of chromosome 18q and prognosis in colorectal cancer. N Engl J Med 1994; 331:213-21.
41. Paik 5, Shak S, Tang G, et al. A multigene assay to predict recurrence of tamoxifen-treated, node-negative breast cancer. N Engl J Med 2004; 351:2817-26.
42. Wang Y, Jatkoe T, Zhang Y, et al. Gene expression profiles and molecular markers to predict recurrence of Dukes' B colon cancer. J Clin Oncol 2004; 22:1564-71.

TABLE 1
Characteristics of Patients with Colorectal Cancer

| | | | | | No. (%) of |
|---|---|---|---|---|---|
| | | | | | Patients With |
| | No. (%) of Patients | | | | Stage III pN1 |
| | pN0 (mol−) | pN0 (mol+) | | P | Disease |
| | (n = 32) | (n = 225) | | Value | (n = 87) |
| Age. y | | | | | |
| <50 | 3 (9.4) | 18 (8.0) | | .25 | 10 (11.5) |
| 50-75 | 24 (75.0) | 140 (62.2) | {close oversize bracket} | | 50 (57.5) |
| >75 | 5 (15.6) | 67 (29.8) | | | 27 (31.0) |
| Sex | | | | | |
| Male | 20 (62.5) | 122 (54.2) | | .38 | 43 (49.4) |
| | | | {close oversize bracket} | | |
| Female | 12 (37.5) | 103 (45.8) | | | 44 (50.6) |
| T stage | | | | | |
| 1/2 | 14 (43.8) | 88 (39.1) | | .32 | 16 (18.4) |
| 3 | 14 (43.7) | 122 (54.2) | {close oversize bracket} | | 50 (57.5) |
| 4 | 4 (12.5) | 15 (6.7) | | | 21 (24.1) |
| Grade | | | | | |
| Well | 2 (6.3) | 17 (7.6) | | .04 | 6 (7.0) |
| Moderate | 20 (62.5) | 178 (79.1) | {close oversize bracket} | | 61 (70.1) |
| Poor/unknown | 10 (31.3) | 30 (13.3) | | | 20 (22.9) |
| Chemotherapy | | | | | |
| Yes | 8 (23.5) | 49 (21.6) | | .68 | 62 (71.3) |
| | | | {close oversize bracket} | | |
| No | 24 (75.0) | 176 (78.2) | | | 25 (28.7) |
| Tumor site | | | | | |
| Left colon | 3 (9.4) | 14 (6.2) | | .84 | 9 (10.3) |
| Right colon | 12 (37.5) | 96 (42.7) | | | 31 (35.6) |
| Sigmoid colon | 13 (40.6) | 84 (37.3) | {close oversize bracket} | | 37 (42.5) |
| Rectum | 4 (12.5) | 31 (13.80) | | | 10 (11.5) |
| No. of lymph | | | | | |
| nodes harvested | | | | | |
| <12 | 11 (34.4) | 34 (15.1) | | .007 | 20 (23.0) |
| | | | {close oversize bracket} | | |
| ≧12 | 21 (65.6) | 191 (84.9) | | | 67 (77.0) |

Abbreviations: GUCY2C, guanylyl cyclase 2C; pN0 (mol−), lymph nodes negative for GUCY2C; pN0 (mol+), lymph nodes positive for GUCY2C (occult metastases).

TABLE 2
Univariate Analysis of Prognostic Factors for Disease Recurrence

| | | Multivariate | | Multivariate | |
|---|---|---|---|---|---|
| | | Hazard Ratio | P | Hazard Ratio | P |
| | | (95% CI) | Value | (95% CI) | Value |
| Parameter | N | LN as Categorical | | LN as Continuous | |
| T Stage | T1/2 | Referent | | Referent | |
| | T3 | 1.75 (0.89-3.43) | 0.106 | 1.85 (0.94-3.64) | 0.076 |
| | T4 | 2.35 (0.67-8.281 | 0.185 | 2.65 (0.76-9.28) | 0.127 |
| Grad | Poor/Unknown | Referent | | Referent | |
| | Well | 0.86 (0.2-3.74) | 0.839 | 0.89 (0.20-3.95) | 0.602 |
| | Moderate | 1.1 (0.42-2.86) | 0.850 | 1.10 (0.42-2.37) | 0.878 |
| Location | Rectal | Referent | | Referent | |
| | Right | 1.09 (0.40-3.03) | 0.861 | 0.97 (0.36-2.60) | 0.948 |
| | Left | 1.52 (0.40-5.86) | 0.541 | 1.43 (0.37-5.45) | 0.602 |
| | Sigmoid | 1.81 (0.71-4.60) | 0.215 | 1.70 (0.67-4.32) | 0.266 |
| LV Invasion | No | Referent | | Referent | |
| | Yes | 0.51 (0.20-1.32) | 0.166 | 0.49 (0.19-1.24) | 0.132 |
| Nodes | <12 | Referent | | | |
| Harvested | >12 | 0.61 (0.31-1.21) | 0.158 | | |
| | Continuous | | | 0.99 (0.97-1.01) | 0.383 |
| Treatment | Surgery | Referent | | Referent | |
| | Surgery + | 1.22 (0.61-2.41) | 0.574 | 1.16 (0.59-2.28) | 0.676 |
| | Chemo | | | | |
| Occult Mets | Mol(−) | Referent | | Referent | |
| | Mol(+) | 4.66 (1.11-19.57) | 0.035 | 4.70 (1.11-19.80) | 0.035 |

TABLE 3
Univariate Analysis of Prognostic Factors for Disease-Free Survival

| | | Multivariate | | Multivariate | |
|---|---|---|---|---|---|
| | | Hazard Ratio | P | Hazard Ratio | P |
| | | (95% CI) | Value | (95% CI) | Value |
| Parameter | N | LN as Categorical | | LN as Continuous | |
| T Stage | T1/2 | Referent | | Referent | |
| | T3 | 1.70 (0.94-3.08) | 0.077 | 1.80 (0.99-3.26) | 0.052 |
| | T4 | 2.98 (1.03-8.61) | 0.043 | 3.27 (1.15-9.33) | 0.027 |
| Grad | Poor/Unknown | Referent | | Referent | |
| | Well | 0.60 (0.15-2.35) | 0.464 | 0.65 (0.16-2.59) | 0.538 |
| | Moderate | 0.98 (0.45-2.12) | 0.952 | 0.99 (0.46-2.16) | 0.984 |
| Location | Rectal | Referent | | Referent | |
| | Right | 1.28 (0.52-3.19) | 0.591 | 1.18 (0.49-2.86) | 0.717 |
| | Left | 1.22 (0.34-4.43) | 0.761 | 1.17 (0.32-4.22) | 0.811 |
| | Sigmoid | 1.74 (0.73-4.43) | 0.208 | 1.63 (0.69-3.87) | 0.266 |
| LV Invasion | No | Referent | | Referent | |
| | Yes | 0.60 (0.27-1.33) | 0.206 | 0.59 (0.27-1.30) | 0.189 |
| Nodes | <12 | Referent | | | |
| Harvested | >12 | 0.65 (0.35-1.22) | 0.181 | | |
| | Continuous | | | 0.99 (0.97-1.01) | 0.223 |
| Treatment | Surgery | Referent | | Referent | |
| | Surgery + | 0.88 (0.47-1.65) | 0.766 | 0.88 (0.47-1.65) | 0.682 |
| | Chemo | | | | |
| Occult Mets | Mol(−) | Referent | | Referent | |
| | Mol(+) | 3.27 (1.15-9.29) | 0.026 | 3.35 (1.17-9.57) | 0.024 |

1. A method of detecting the level of GCC mRNA present in a tissue sample using quantitative (q) RT-PCR comprising the steps of:
a) isolating RNA from one or more tissue samples obtained from an individual;
b) performing quantitative RT-PCR on at least a sample of the RNA using the primers that amplify GCC;
c) performing quantitative RT-PCR on at least a sample of the RNA using the primers that amplify a reference marker; and
d) estimating by logistic regression analysis of amplification profiles from the quantitative RT-PCR reactions to provide an efficiency-adjusted relative quantification based on parameter estimates from fitted models.

2. The method of claim 1 further comprising
e) comparing the efficiency-adjusted relative quantification to an established cut off.

3. The method of claim 1 wherein the efficiency-adjusted relative quantification is used to determine if a tissue sample contains GCC mRNA indicative of occult metastasis.

4. The method of claim 1 wherein the established cut off is the median of efficiency-adjusted relative quantifications compiled from a plurality of samples from a plurality of individuals.

5. The method of claim 1 comprising performing quantitative RT-PCR to amplify GCC mRNA using primers ATTCTAGTGGATCTTTTCAATGACCA (SEQ ID NO:1) and CGTCAGAACAAG-GACATTTTTCAT (SEQ ID NO:2).

6. The method of claim 1 comprising performing quantitative RT-PCR using a Taqman probe (FAM-TACTTGGAGGACAATGTCACAG-CCCCTG-TAMRA) (SEQ ID NO:3).

7. The method of claim 1 wherein the reference marker is beta actin.

8. The method of claim 1 wherein the reference marker is beta actin and further comprising performing quantitative RT-PCR to amplify beta actin mRNA using primers CCACACTGTGCCCATCTACG (SEQ ID NO:4) and AGGATCTTCATGAG-GTAGTCAGTCAG (SEQ ID NO:5).

9. The method of claim 1 wherein the reference marker is beta actin and further comprising performing quantitative RT-PCR using a Taqman probe (FAM-ATGCCC-X(TAMRA)-CCCCCATGCCATCCTGCGTp) (SEQ ID NO:6).

10. The method of claim 1 wherein the sample is from a patient diagnosed with primary colorectal cancer, gastric or esophageal cancer.

11. The method of claim 1 wherein the sample is a lymph node sample.

12. The method of claim 1 wherein 2, 3, 4, 5, 6, 7, 8, 9, 10, 11, 12 or more samples are obtained from the patient.

13. The method of claim 1 wherein 2, 3, 4, 5, 6, 7, 8, 9, 10, 11, 12 or more lymph node samples obtained from the patient.

14. The method of claim 1 wherein the data is used to determine risk of recurrence.

15. The method of claim 1 wherein the patient has been diagnosed with esophageal dysplasia, esophageal lesion or other abnormal esophageal.

16. The method of claim 15 wherein the sample is an esophageal tissue sample.

17. A composition comprising primers ATTCTAGTGGATCTTTTCAATGACCA (SEQ ID NO:1) and CGTCAGAACAAG-GACATTTTTCAT (SEQ ID NO:2).

18. The composition of claim 17 further comprising Taqman probe (FAM-TACTTGGAGGACAATGTCACAG-CCCCTG-TAMRA) (SEQ ID NO:3).

19. The composition of claim 17 further comprising CCACACTGTGCCCATCTACG (SEQ ID NO:4) and AGGATCTTCATGAG-GTAGTCAGTCAG (SEQ ID NO:5).

20. The composition of claim 17 further comprising Taqman probe (FAM-ATGCCC-X(TAMRA)-CCCCCATGCCATCCTGCGTp) (SEQ ID NO: 6).

21. A kit comprising a first container comprising the composition of claim 17 and a second container comprising Taqman probe (FAM-TACTTGGAGGACAATGTCACAG-CCCCTG-TAMRA) (SEQ ID NO:3).

22. The kit of claim 21 further comprising primers CACACTGTGCCCATCTACG (SEQ ID NO: 4) and AGGATCTTCATGAG-GTAGTCAGTCAG (SEQ ID NO: 5).

23. The kit of claim 22 further comprising Taqman probe (FAM-ATGCCC-X(TAMRA)-CCCCCATGCCATCCTGCGTp) (SEQ ID NO: 6).

24. The kit of claim 21 further comprising instructions for programming a device to estimate by logistic regression analysis of amplification profiles from quantitative RT-PCR reactions, efficiency-adjusted relative quantifications based on parameter estimates from fitted models; wherein said instructions are copied to a fixed medium.

25. The kit of claim 21 further comprising instructions for programming a device to compare an efficiency-adjusted relative quantification with established cut off points in order to determine if a sample that was used to produce the efficiency-adjusted relative quantification contained a level of GCC mRNA exceeding a specific threshold.

26. A composition comprising CCACACTGTGCCCATCTACG (SEQ ID NO:4) and AGGATCTTCATGAG-GTAGTCAGTCAG (SEQ ID NO:5).

27. The composition of claim 26 further comprising (FAM-ATGCCC-X(TAMRA)-CCCCCATGCCATCCTGCGTp) (SEQ ID NO:6).

28. A system for quantifying GCC encoding mRNA by quantitative (q) RT-PCR comprising a device programmed to estimate by logistic regression analysis of amplification profiles from quantitative RT-PCR reactions to produce an efficiency-adjusted relative quantification based on parameter estimates from fitted models.

29. The system of claim 28 wherein the device is programming to compare an efficiency-adjusted relative quantification with established cut off points in order to determine if a sample that was used to produce the efficiency-adjusted relative quantification contained a level of GCC mRNA exceeding a specific threshold.

30. A method of determining the level of GCC mRNA present in a tissue sample using quantitative (q) RT-PCR comprising the steps of:
a) isolating RNA from one or more tissue samples obtained from an individual;
b) performing quantitative RT-PCR on at least a sample of the RNA using the primers that amplify GCC using primers ATTCTAGTGGATCTTTTCAATGACCA (SEQ ID NO:1) and CGTCAGAACAAG-GACATTTTTCAT (SEQ ID NO:2).

31. The method of claim 30 comprising performing quantitative RT-PCR using a Taqman probe (FAM-TACTTGGAGGACAATGTCACAG-CCCCTG-TAMRA) (SEQ ID NO:3).
